(19) United States
(12) Patent Application Publication
(10) Pub. No.: US 2012/0016475 A1
(43) Pub. Date: Jan. 19, 2012

(54) DEGRADABLE COMPOSITE

(75) Inventors: Andrew James Parsons, Nottingham (GB); John Derek Irvine, Nottingham (GB)

(73) Assignee: The University of Nottingham, Nottingham (GB)

(21) Appl. No.: 13/139,983

(22) PCT Filed: Dec. 14, 2009
(86) PCT No.: PCT/GB09/02882

§ 371 Date: Oct. 4, 2011

(30) Foreign Application Priority Data
Dec. 16, 2008 (GB) 0822892.6

(51) Int. Cl. A61F 2/28; A61P 19/00; A61F 2/02; A61K 9/00

(52) U.S. Cl. 623/16.11; 424/423; 623/23.72

(57) ABSTRACT

The invention relates to degradable composites and their use in biomedical implants, in particular the repair of damaged bones and/or cartilage.

The present invention relates to degradable composites and their use in biomedical implants.

### BACKGROUND TO THE DISCLOSURE

Degradable composites have attracted widespread attention during the last few decades due to their increasing application in biomedical fields, and to the search for degradable substitutes for use as packaging.

In the biomedical field the interest lies predominantly in the production of, and use of, composites comprising polymers which once implanted in the body will degrade over time. Preferably the body can resorb the degradation products produced when the polymer degrades. To exemplify the role of degradable polymer composites in the biomedical field, it is noted that such composites have been used as bone implants, preferably to provide a temporary repair to damaged bone. The implants may be in the form of pins, plates or custom shaped implants. Preferably the composites used are strong enough to support bone as it heals, have good biocompatibility and can be molded to a desired shape. In the biomedical area the need for a composite is related to the need to modify the physical/chemical properties of the pure polymer matrix in order to make it fit for purpose within the particular application.

With regard to packaging material, it is becoming ever more important to devise materials for packaging conventional commodities that will degrade. This helps to solve the problem of increasing levels of discarded non-degradable plastic waste in landfills. Accordingly, increasing efforts are being put into developing degradable polymer composites suitable for use as packaging materials.

This disclosure relates to polymer composites with improved degradation properties. This improvement in degradation properties may be evidenced by providing some control over the rate of degradation, so that composites can be designed to degrade over specific time periods or profiles.

The disclosure also provides a composite comprising a degradable matrix material, a degradable filler material and an interface agent or agents intended to improve contact and/or adhesion between matrix and filler. The nature of this filler is such that its inclusion should impart additional processing or material properties to the final composite that the ‘neat’ polymer matrix does not possess alone. For example the filler may (a) reduce process issues related to polymerisation/post reaction exotherm (b) aid in the definition of degradation rates of the polymer (c) impart additional mechanical strength so that the material can be used in load bearing structures.

### STATEMENTS OF INVENTION

According to an aspect of the invention there is provided a composite suitable for use in a biodegradable implant comprising:
i) a matrix comprising one or more polymers;
ii) a filler comprising one or more polymers; and
iii) an interface agent comprising a functionalised polymer wherein said interface agent contacts at least part of said matrix and/or filler.

In a preferred embodiment of the invention the interface agent comprises a polymer selected from the group consisting of: polyethylene glycol, polyhydroxy acids, poly lactic acid, polycaprolactone, polyglycolic acid and/or co-polymers or mixtures thereof

In a preferred embodiment of the invention said polymer is poly lactic acid.

In a preferred embodiment of the invention said polymer is functionalized wherein said polymer is modified by the addition of one or more: hydroxyl groups, carboxylic acid groups, esters, organic salts, inorganic salts [e.g. Li, K, Ca, Mg salts], amines [e.g. NH_{[x]}], thiols [e.g. SH groups], amides, amino groups, urethane, sorbitol or mixtures thereof.

In a preferred embodiment of the invention said inorganic salt is sodium. Preferably said functionalised polymer is end functionalised, preferably sodium salt ended.

In a preferred embodiment of the invention said polymer is functionalized by addition of one or more hydroxyl groups.

Preferably, polylactic acid is functionalized by addition of 1-5 hydroxyl groups. Preferably, 5 hydroxyl groups.

In a preferred embodiment of the invention said matrix comprises one or more polymers selected from the group consisting of: acrylics, polyesters, polyolefins, polyurethanes, silicon polymers, vinyl polymers, halogenated hydrocarbons such as Teflon™, nylons, proteinaceous materials, and copolymers and combinations thereof.

In a preferred embodiment of the invention said matrix comprises one or more polymers selected from the group consisting of: polyorthoester made from polylactides, poly lactic acids (PLA, PLLA, PDLLA), epsilon caprolactone, polycaprolactone (PCL), polyglycolic acid (PGA), polypropylene fumarate, polycarbonates such as polymethyl carbonate and polytrimethylenecarbonate, polyiminocarbonate, polyhydroxybutyrate, polyhydroxyvalerate, polyoxalates such as poly(alkylene)oxalates, polyamides such as polyesteramide and polyanhydrides, and copolymers and combinations thereof

In a preferred embodiment of the invention said matrix comprises poly lactic acid.

In a further preferred embodiment of the invention said filler is carbon based, glass, for example phosphate glass, bioglass, ceramic, aramide, natural materials such as jute and hemp, polyethylene, polyamide.

According to an aspect of the invention there is provided a composite suitable for use in a biodegradable implant comprising:
i) a matrix comprising a poly lactic acid polymer;
ii) a filler comprising phosphate glass; and
iii) an interface agent comprising a functionalised poly lactic acid polymer wherein said interface agent contacts at least part of said matrix and/or filler.

According to an aspect, the invention provides a biodegradable medical implant comprising a composite according to the invention.

The medical implant may be a pin, plate or custom shaped implant.

The medical implant may be used in transplant surgery, bone resurfacing, the fixation of fractures and/or tissue scaffolding. The medical implant may be used in cranio-facial or maxillo-facial surgery, or in orthopaedic surgery such as the replacement of bone, cartilage and/or meniscus material.

According to an aspect of the invention there is provided a composite according to the invention for use in the manufacture of a medical implant.

According to a further aspect of the invention there is provided a composite according to the invention for use in the treatment of damaged bones and/or cartilage.

According to a further aspect of the invention there is provided a method to repair bone and/or cartilage damage comprising surgically inserting a composite according to the invention into a subject in need of treatment.

In a preferred method of the invention said subject is human.

In an alternative preferred method of the invention said subject is a non-human animal.

Preferably said non-human animal is a domestic pet, e.g. a cat or dog. Alternatively said non-human animal is a horse, cow or sheep.

According to a further aspect, the invention provides a film or other packaging material comprising a composite according to the invention. The film and/or other packaging material may have degradation rates tailored to out last the shelf life of the packaged goods but to ensure full biodegradation a short time thereafter.

According to another aspect, the invention provides a film or other gel material comprising a composite according to the invention. The film and/or gel material may have degradation rates and polymer film and/or gel properties such that it acts as a biodegradable wound dressing. In such an application the film and/or gel material could be applied as part of a spray or a preformed ‘strip’ structure. It may also contain fillers such as TiO2 to protect the wound tissue from exposure to, for example, UV radiation.

### DEFINITIONS

The agent or agents are a substance or mixture of substances designed to improve contact and/or adhesion between matrix and filler whilst also being degradable, with their rate of degradation being matched suitably to the matrix and/or filler.

The agent or agents may be but are not limited to functionalised polymers. The polymers may be of any molecular weight. The polymers may be but are not limited to; polyethylene glycol, polyhydroxy acids, poly lactic acid, polycaprolactone, polyglycolic acid and/or co-polymers or mixtures thereof. The polymers may be multi-functional. The functionality or functionalities may be but are not limited to; hydroxyl, carboxylic acid, ester, organic salt, inorganic salt, amine, thiol, amide, amino, urethane or mixtures thereof. These functional group(s) may be end functional or main chain functional and may be part of a statistical co-polymer or as part of a single block, graft or arm of complex architectural co-polymer such as A-B or A-B-A block co-polymer; graft co-polymer, star co-polymer, hyper-branched co-polymer, dendritic co-polymer. This may take the form of a single functionality at a particular site in the specifically designed molecular structure of the interfacial agent or as a multi-functional segment/block/head group in the specifically designed interfacial agent macro co-polymer structure.

The agent or agents may interact with the matrix and/or filler via mechanical or chemical means including but not limited to; Van der Waals interaction, ionic bonding, covalent bonding, dative bonding, pi-bonding, other means of chemical attachment and/or mechanical interlock. The agent or agents may be applied to the matrix or to the filler in the first instance. The agents may undergo chemical reaction with the filler and/or matrix prior to or subsequent to the combination of filler and matrix.

Preferably the interface agent or agents are applied to the filler(s) prior to combination of the filler(s) and matrix but also may be added to the matrix and filler blend to form in-situ bonds to the filler surface after migration through the matrix to the filler surface.

Not all of a particular filler needs to be treated with an interface agent

Not all of the types of filler need to be treated with an interface agent

Not all of the filler needs to interact with the interface agent(s) via in-situ methods, post the mixing of all the system components

Not all of the matrix needs to interact with the interface agent(s) via in-situ methods post the mixing of all the system components.

The interface agent(s) may be mixed with the monomer or oligomer prior to, or during, polymerisation.

Alternatively, the interface agent(s) may be added to the composite after polymerisation, for example, it may be added by melt mixing, powder mixing, mixing in solution and/or in monomer mixture.

A composite according to the invention may comprise homogenous polylatic acid (PLA) as the matrix, phosphate glass as the filler and a functionalised oligomeric/polymeric PLA as an interface agent, where the functionality is such that it will exhibit a strong preference to be in intimate contact with or actually bond to the phosphate glass through means noted previously.

A composite according to the invention may comprise homogeneous PLA, phosphate glass and a functionalised oligomeric/polymeric PLA has an improved interfacial contact such that it has material properties equal to or greater than those of a homogeneous PLA and phosphate glass composite without the presence of an interface agent. Additionally, the interfacial agent functionality should also, preferably both match the decomposition/degradation rates of the lower molecular weight interface agent and the matrix property such that the interfacial agent acts to maintain these properties during degradation and provide control over the rate of loss of the overall composite material and thus its mechanical properties. The larger number of chain ends in such a lower molecular weight polymeric interface agent should lead to increased levels of polymeric degradation of the interface agent, which in turn may release decomposition products that will lead to the accelerated decomposition of the matrix polymer. Furthermore, the functionality of the interface agent may be of a type which would contain groups which may be thought likely to increase the degradation rate of the lower molecular weight interface agent. For example the functionality may be hygroscopic or hydrophilic to a greater or lesser degree and/or present as part of an oligomer which is hydrolytically unstable.

The composite may comprise but is not limited to one or more polymers selected from the group comprising acrylics, polyesters, polyolefins, polyurethanes, silicon polymers, vinyl polymers, halogenated hydrocarbons such as Teflon™, nylons, proteinaceous materials, and copolymers and combinations thereof. For example, the composite may comprise a polyorthoester made from polylactides, poly lactic acids (PLA, PLLA, PDLLA), epsilon caprolactone, polycaprolactone (PCL), polyglycolic acid (PGA), polypropylene fumarate, polycarbonates such as polymethyl carbonate and polytrimethylenecarbonate, polyiminocarbonate, polyhydroxybutyrate, polyhydroxyvalerate, polyoxalates such as poly(alkylene)oxalates, polyamides such as polyesteramide and polyanhydrides, and copolymers and combinations thereof.

The composite may comprise polymers and/or copolymers of aliphatic polyesters, such as poly-ε-caprolactone and/or biocompatible derivatives and analogues thereof.

Preferably the composite comprises a thermoplastic polymer and/or copolymer.

A composite may further comprise one or more other polymer and/or copolymer phase(s). The other polymer and/or copolymer phases may be included using a method such as, but not limited to, blending, water based processing such as emulsion/suspension/dispersion, solution processing or monomer processing. The additional polymers and/or copolymers may be degradable or biodegradable.

Preferably, whatever the delivery route for the interface agent(s), the composition of the invention has a controlled rate of degradation, which may or may not be matched to the main matrix polymer or co-polymer's degradation rate, dependent upon the actual application.

The filler(s) may be of any material including but not limited to; carbon, glass, ceramic, aramide, natural materials such as jute and hemp, polyethylene, polyamide.

The filler(s) may be of any physical form including but not limited to; irregular or regular shaped particles, rods, discs/plates, cylinders, tubes and/or fibres which may be in the form of a random or regular mesh, woven or non-woven inserts or a three dimensional structure.

The filler may be a mixture of materials of similar composition but different physical form. The filler may be a mixture of materials of different composition but similar physical form. The filler may be a mixture of materials of different composition and different physical form.

The filler may be added to provide increased physical properties including but not limited to; strength, modulus, toughness and hardness.

The polymer and/or copolymer and/or filler and/or interface agent(s) may be biodegradable. Preferably, the entire composite is biodegradable.

The polymer and/or co-polymer and/or filler and/or interface agent(s) may provide an improvement to processing, for example by improving control of flow rate, improving complete filling of the mould, improving wet-out of filler or providing control of the reaction exotherm.

The composite components (filler(s), matrix and interface agent(s)) may degrade at the same or similar rates.

Some of the components of the composite may degrade at different rates.

The composite may be produced by any standard method of production; such as compression moulding, resin injection, cell casting, extrusion or monomer transfer moulding. The composite may be additionally formed after production by for example thermoforming

The polymer and/or copolymer matrix may be produced with or without the use of a catalyst, initiator or accelerant.

The interface agent may be produced with or without the use of a catalyst, initiator or accelerant.

Any suitable catalysts may be used

The catalyst may be stannous octoate.

The composite may further comprise more than one polymer and/or copolymer.

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

An embodiment of the invention will now be described by example only and with reference to the following FIGURE, materials, methods and examples:

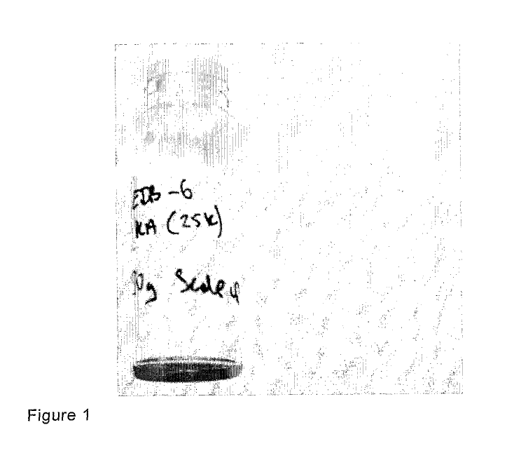

FIG. 1 illustrates sodium salt ended PLA after a grinding cycle.

### EXAMPLE 1

### Effect of Functionality of Interface Agent on the Interfacial Shear Strength when Used to Couple Phosphate Glass Fibre with PLA Polymer

Natureworks PLA 3051D (10 g) was placed in a 100 mL round bottomed flask equipped with a magnetic stirrer. The polymer was heated to 175-185° C. for 1.30 hours under nitrogen atmosphere. Sorbitol (2.108 g—molar ratio of 1 sorbitol to 12 PLA) and tin (II) 2-ethylhexanoate catalyst (0.054 g) were then added into the molten PLA. The total mixture was held at 170° C. and stirred for 15 hours under a nitrogen atmosphere. After this time period the total mixture was decanted whilst hot onto aluminium foil and allowed to cool to room temperature. The resulting product was a brittle, amber coloured, transparent glassy solid. The reaction mixture was then dissolved in dichloromethane (DCM) and precipitated in excess methanol, filtered and dried under vacuum. The final product of sorbitol-ended PLA having Mn=2881, PDI=3.2 showed peak in ¹H NMR at δ 1.58 (CH3, m), 5.19 (CH, m).

The above mentioned protocol and the ratios were followed for the synthesis of ethylene glycol (EG) and glycerol ended PLA material. The amount of EG and Glycerol were used (1.068 g, 11.59 mmol) and (0.72 g, 11.59 mmol) respectively. The Mn and PDI of the resultant Ethylene glycol and Glycerol ended PLA was obtained 1217, 2.71 and 8210, 1.98 respectively. The H-NMR peaks were found at δ 1.58 (CH3, m), 5.20 (CH, m) and δ 1.59 (CH3, m), 5.19 (CH, m) for Ethylene glycol and Glycerol ended PLA respectively.

The materials used to produce these oligomers were chosen such that the functionality at the end of the oligomer chain provided one (using Ethylene glycol), two (using glycerol) or five (using sorbitol) hydroxyl groups that would be expected to bond to the fibre surface through a condensation reaction as follows:

Fibres were soaked in a solution of oligomer dissolved in choloform for 30 minutes with a fiber:solution ratio of 1.5 g:100 ml and a sizing agent:solvent ratio of 0.0043 moles:100 ml. The fibres were then removed from the solution and dried for 2 hours at room temperature before curing in an oven at 230° C. for 3-4 hours. The fibres were then left to cool for 24 hours before soaking in chloroform to remove any excess unreacted oligomer. The fibres were then dried in an oven at 120° C. for 2 hours.

IFSS values of the specimens were obtained using the single fibre fragmentation test. The matrix used for the IFSS studies was Natureworks PLA (grade 3051D, Natureworks LLC, U.S.A). Thin films (approx. 0.2 mm thick) were prepared by compression moulding 5 g of PLA pellets between PTFE lined aluminium platens. Samples were pressed at 210° C. and 3-4 bar for 30 seconds before immediately cooling under pressure. The films were cut into 80×20 mm specimens. To produce single fibre composite (SFC) specimens for testing, a single fibre was aligned axially between two rectangular pieces and held in place using adhesive tape. Three such assemblies were placed in a mould containing rectangular hollow shapes (80×20×0.4) mm³. The mould was heated at 210° C. for 5 minutes, followed by pressing with a 2.5 kg weight for 1 minute, after which the mould was then cooled to room temperature. The resulting specimens were finally cut using a dog bone cutter. These were axially loaded in a tensile testing machine (Hounsfield series S testing, U.K.) with a load cell of 1 kN and crosshead speed of 1 mm/min. All IFSS values were obtained from an average of 5-10 repeat specimens. After having conducted the tensile tests, the specimens were placed under an optical microscope (Nikon Optiphot, Japan) and the number of fibre fragments that were present in the 25 mm gauge length was tallied, in order to calculate the IFSS.

The most commonly used method to calculate IFSS is the Kelly-Tyson model, which shows that

τi=d·σf/2Lc

Where τi is the IFSS, d is the fibre diameter, σf is the single fibre tensile strength at the critical fragment length Lc.

σf=σo(Lc/L0)−1/m

Lc=4/3(Lf)

Lf=L0/N

Where σo and m are the Weibull scale and shape parameter respectively, for the fibre strength at gauge length L0, Lf is the average fragment length, N is the number of fibre fragments obtained from the SFC tests.

| Oligomer used | IFSS value |
|---|---|
| None (control) | 9 ± 3 |
| Ethylene glycol functionalised | 9 ± 3 |
| Glycerol functionalised | 17 ± 3 |
| Sorbitol functionalised | 23 ± 10 |

It's clearly established that the oligomer functionality affects the interfacial bonding between the fibre and the matrix and that this is dependent on the number of functional groups.

### EXAMPLE 2

### Degradative Resistance of Example Functionalised Oligomer Intended for Use as an Interface Agent

Sodium Ended PLA from Dilactide:

Dried lactide (5 g, 35 mmol) and lactic acid sodium salt (0.112 g, 1 mmol) were added to a 25 mL round bottomed flask equipped with a magnetic stirrer. The mixture was heated to 140° C. and stirred for 24 hours under a nitrogen atmosphere. After this time period the total mixture was decanted whilst hot onto aluminium foil and allowed to cool to room temperature. The resulting product was a brittle, amber coloured, transparent glassy solid (4.63 g, 91%): ¹H NMR δ 1.56 (CH3, m), 5.26 (CH, m). T_{g} (° C.): inflection 47.03, Mn=4410, PDI=2.33.

Acid Ended PLA from Dilactide:

Dried lactide (5 g, 35 mmol) and lactic acid (0.105 g, 1 mmol) were added to a 25 mL round bottomed flask equipped with a magnetic stirrer. The mixture was heated to 130° C. for 48 hours under a nitrogen atmosphere. After this time period the total mixture was decanted whilst hot onto aluminium foil and allowed to cool to room temperature. The resulting product was a very light brown, transparent, hygroscopic solid (4.57 g, 90%): ¹H NMR δ 5.28 (CH, m), 1.59 (CH3, m). T_{g} (° C.): inflection 30.95, Mn=1419, PDI=1.67.

### Acid Value Analysis

Acid values of produced materials were determined via titration. A known weight of polymer was dissolved in indicator solution (phenolphthalein (1% w/w) in 3:2 toluene:propan-2-ol solution). This was then titrated against a potassium hydroxide standard (0.110M, in a 4:1 propan-2-ol:water solution) until the end-point was determined via a sharp change in colour. All titrations were performed in duplicate and results calculated using the modified Stockmeier equation:

$$\frac{mMT}{w} = A$$

m=relative molecular mass of base (KOH) used (g)
M=molarity of the titre (mol/dm−3)
T=total amount of titre used (mL)
w=sample weight (g)
A=acid value (mg KOH g−1)

| | | Average Acid Value (mg KOH g⁻¹) | | |
|---|---|---|---|---|
| Time since made | | Bulk tin catalysed | Acid ended | Sodium ended |
| 0 | days | 57* | 62.74 | 5.53 |
| 2 | days | | 90.81 | — |
| 16 | weeks | | 241.80 | 26.54 |

*Value taken from Donald Garlotta, Journal of Polymers and the Environment, Volume 9, Number 2, Pages 63-84

Acid value is a means of quantifying the end groups in a polymer. For PLA this value increases as it degrades, since each scission of a PLA polymer chain by a water molecule will provide another acid end-group, therefore increasing the acid value. It can be seen that the acid ended oligomer has a similar acid value to the bulk PLA and that this value increases significantly over time, indicating a rapid degradation of the oligomer. However, the oligomer protected with a sodium functionality significantly retarded the degradation of the oligomer, remaining a free-flowing powder. In this manner the oligomer degradation rate is slowed and could be matched to the matrix degradation rate.

### EXAMPLE 3

### Polymer Processing Grinding Trials

In-order to be used in the application, these prills must be processed into a powder once purchased. However, successfully grinding these prills has proved to be exceptionally difficult. This problem is thought to be related to the glass transition temperature (Tg) and/or the molecular weight of the material. The Tg, measured by Differential Scanning calorimetry (DSC), was found to be in the range 40-60° C. and so it is possible that as soon as the grinding begins the temperatures within the processing equipment will rises above the materials Tg leading to it “smearing”.

It was proposed that by synthesising materials with a low molecular weight, the polymer chain entanglement would be much lower than in the high molecular weight PLA and therefore affording easier grinding (see oligomer structures in FIG. 4). It was found that both the sodium ended and acid ended low molecular weight PLA chains were highly susceptible to grinding. However, the acid ended PLA was observed to be exceptionally hygroscopic and became saturated with water from the atmosphere within hours of grinding. In this case the ground material reformed into a single tacky mass of PLA and thus was deemed unsuitable for use in the drilling muds by Cleansorb. The sodium salt ended PLA did not display this characteristic, the powder formed was very fine and remained in this state indefinitely. The powder produced in via the use of a hand grinder is shown in FIG. 1

1. A composite suitable for use in a biodegradable implant comprising:
i) a matrix comprising one or more polymers;
ii) a filler comprising one or more biodegradable polymers; and
iii) an interface agent comprising a functionalised polymer wherein said interface agent contacts at least part of said matrix and/or filler.

2. A composite according to claim 1 wherein the interface agent comprises a polymer selected from the group consisting of: polyethylene glycol, polyhydroxy acids, poly lactic acid, polycaprolactone, polyglycolic acid and/or co-polymers or mixtures thereof

3. A composite according to claim 2 wherein said polymer is poly lactic acid.

4. A composite according to claim 1 wherein said functionalized polymer is modified by the addition of one or more: hydroxyl groups, carboxylic acid groups, esters, organic salts, inorganic salts (e.g. Li, K, Ca, Mg salts), amines (e.g. NH₍ₓ₎), thiols (e.g. SH groups), amides, amino groups, urethane, sorbitol or mixtures thereof.

5. A composite according to claim 4 wherein said polymer is modified by addition of sodium.

6. A composite according to claim 1 wherein said functionalised polymer is end functionalised.

7. A composite according to claim 6 wherein said polymer is sodium salt ended.

8. A composite according to claim 1 wherein said polymer is functionalized by addition of one or more hydroxyl groups.

9. A composite according to claim 8 wherein said polymer is functionalized by addition of 2-5 hydroxyl groups.

10. A composite according to claim 2 wherein said polymer is poly lactic acid.

11. A composite according to claim 1 wherein said matrix comprises one or more polymers selected from the group consisting of: acrylics, polyesters, polyolefins, polyurethanes, silicon polymers, vinyl polymers, halogenated hydrocarbons such as Teflon™, nylons, proteinaceous materials, and copolymers and combinations thereof.

12. A composite according to claim 11 wherein said matrix comprises one or more polymers selected from the group consisting of: polyorthoester made from polylactides, poly lactic acids (PLA, PLLA, PDLLA), epsilon caprolactone, polycaprolactone (PCL), polyglycolic acid (PGA), polypropylene fumarate, polycarbonates such as polymethyl carbonate and polytrimethylenecarbonate, polyiminocarbonate, polyhydroxybutyrate, polyhydroxyvalerate, polyoxalates such as poly(alkylene)oxalates, polyamides such as polyesteramide and polyanhydrides, and copolymers and combinations thereof

13. A composite according to claim 12 wherein said matrix comprises poly lactic acid.

14. A composite according to claim 1 wherein said filler is carbon based, glass, phosphate glass, bioglass, ceramic, aramide, jute and hemp, polyethylene, polyamide.

15. A composite according to claim 14 wherein said filler is phosphate glass.

16. A composite suitable for use in a biodegradable implant comprising:
i) a matrix comprising a poly lactic acid polymer;
ii) a filler comprising phosphate glass; and
iii) an interface agent comprising a functionalised poly lactic acid polymer wherein said interface agent contacts at least part of said matrix and/or filler.

17. A composite according to claim 16 wherein said interface agent is functionalised.

18. A composite according to claim 16 wherein said interface agent is poly lactic acid and is sodium salt ended.

19. A composite according to claim 16 wherein poly lactic acid is functionalized by addition of one or more hydroxyl groups.

20. A composite according to claim 19 wherein poly lactic acid is functionalized by addition of 2-5 hydroxyl groups.

21. A composite according to claim 20 wherein poly lactic acid is functionalised by addition of at least 5 hydroxyl groups.

22. A biodegradable medical implant comprising a composite according to claim 1 or 16.

23. (canceled)

24. (canceled)

25. A method to repair bone and/or cartilage damage comprising surgically inserting a composite according to claim 1 or 16 into a subject in need of such treatment.

26. A method according to claim 25 wherein said subject is a member selected from a human and a non-human animal.

27. (canceled)
